(19) United States
(12) Patent Application Publication
(10) Pub. No.: US 2023/0021211 A1
(43) Pub. Date: Jan. 19, 2023

(54) PERITONEAL DIALYSIS CASSETTE MOUNTING CHAMBER

(71) Applicant: Medtronic, Inc., Minneapolis, MN (US)

(72) Inventors: Rathnakara Narayana, Bangalore (IN); Rajkumar Vp, Dindigul (IN)

(73) Assignee: Medtronic, Inc, Minneapolis, MN (US)

(21) Appl. No.: 17/374,231

(22) Filed: Jul. 13, 2021

(51) Int. Cl. A61M 1/28

(52) U.S. Cl. CPC A61M 1/28; A61M 1/282; A61M 2205/3344

(57) ABSTRACT

A peritoneal dialysis cassette and an interface between the cassette and a peritoneal dialysis system is provided. The peritoneal dialysis cassette can include one or more fluid passages connecting a plurality of inlet/outlet ports, with one or more linear actuators usable to selectively direct fluid through the peritoneal dialysis cassette.

### FIELD

A peritoneal dialysis cassette and an interface between the cassette and a peritoneal dialysis system is provided. The peritoneal dialysis cassette can include one or more fluid passages connecting a plurality of inlet/outlet ports, with one or more linear actuators usable to selectively direct fluid through the peritoneal dialysis cassette.

### BACKGROUND

Conventional peritoneal dialysis systems often use a cassette to direct and control fluid movement for the generation of peritoneal dialysis fluid and delivery of peritoneal dialysis therapy. Often, the known systems use balloon actuators to occlude passages through the cassette. Conventional balloon actuators expand and contract within the fluid passages of the cassette. However, using balloon actuators typically requires an airtight seal between the balloon actuators and throughout the cassette. As such, the available cassettes typically require a thin, tough, and elastic surface capable of forming a seal while repeatedly underdoing cyclic pressures. Such features and membrane requirements can be challenging and expensive. As such, there is a need for systems, methods, and components that can use a mechanical force to occlude passages rather than using air. The need extends to suitable pumping systems to control valves and direct flow that do not require an airtight seal. The need extends to cassettes and valves that can operate to selectively direct fluid through a cassette using linear actuation.

### SUMMARY OF THE INVENTION

The problem to be solved by the present invention is the movement of fluid through a peritoneal dialysis cassette for any functions performed with a peritoneal dialysis system. The solution is to include one or more linear actuators to occlude selected passages through the peritoneal dialysis cassette and to control the linear actuators to selectively direct fluid throughout the peritoneal dialysis system.

The first aspect relates to a peritoneal dialysis cassette. In any embodiment, the peritoneal dialysis cassette can include a rigid surface; a membrane surface sealed to the rigid surface; the peritoneal dialysis cassette defining one or more fluid passages in between the membrane surface and the rigid surface; two or more inlet/outlet ports fluidly connected to the one or more fluid passages; the one or more fluid passages aligned with one or more linear actuators; the one or more linear actuators positioned to occlude at least one of the one or more fluid passages in a closed state and to allow fluid movement through the one or more fluid passages in an open state.

In any embodiment, the one or more linear actuators can selectively direct fluid through the one or more fluid passages from a first specified inlet/outlet port to a second specified inlet/outlet port.

In any embodiment, the rigid surface is sealed to the membrane surface by pressure sealing or clamping.

In any embodiment, the one or more fluid passages can include at least a first fluid passage fluidly connecting a first inlet/outlet port to a second inlet outlet port and at least a second fluid passage fluidly connecting the first inlet/outlet port to a third inlet/outlet port.

In any embodiment, at least one of the linear actuators can be positioned to selectively direct fluid from the first inlet/outlet port to either the second inlet/outlet port or the third inlet/outlet port.

In any embodiment, at least one of the linear actuators can be positioned to selectively direct fluid from either the second inlet/outlet port or the third inlet/outlet port to the first inlet/outlet port.

In any embodiment, at least a first inlet/outlet port and a second inlet/outlet port can be fluidly connectable to a pump.

In any embodiment, each of the linear actuators can include a pinch bud in contact with the membrane surface in the closed state.

The features disclosed as being part of the first aspect can be in the first aspect, either alone or in combination, or follow any arrangement or permutation of any one or more of the described elements. Similarly, any features disclosed as being part of the first aspect can be in a second or third aspect described below, either alone or in combination, or follow any arrangement or permutation of any one or more of the described elements.

The second aspect relates to a system. In any embodiment, the system can include the peritoneal dialysis cassette of the first aspect, and a peritoneal dialysis cycler; the peritoneal dialysis cycler having a pneumatic system; the pneumatic system controlling the one or more linear actuators to be in an open or a closed state.

In any embodiment, the system can include a control system; the control system in communication with the pneumatic system to selectively direct fluid through the one or more fluid passages.

In any embodiment, the one or more linear actuators can include at least two linear actuators.

In any embodiment, the peritoneal dialysis cycler can include an air cushion chamber; the air cushion chamber in contact with the rigid surface of the peritoneal dialysis cassette.

In any embodiment, the peritoneal dialysis cycler can include a patient line fluidly connectable to a catheter; the patient line fluidly connectable to a first inlet/outlet port of the peritoneal dialysis cassette; and a second inlet/outlet port of the peritoneal dialysis cassette can be fluidly connectable to a peritoneal dialysis fluid bag; the first inlet/outlet port and second inlet/outlet port fluidly connectable through a first fluid passage; wherein a first linear actuator is positioned to either occlude or allow fluid to pass through the first fluid passage.

In any embodiment, the peritoneal dialysis cycler can include a patient line fluidly connectable to a catheter; the patient line fluidly connectable to a first inlet/outlet port of the peritoneal dialysis cassette; and a second inlet/outlet port of the peritoneal dialysis cassette can be fluidly connectable to a drain line; the first inlet/outlet port and second inlet/outlet port fluidly connectable through a first fluid passage; wherein a first linear actuator is positioned to either occlude or allow fluid to pass through the first fluid passage.

In any embodiment, the peritoneal dialysis cycler can include at least one peritoneal dialysis fluid source fluidly connected to a first inlet/outlet port of the peritoneal dialysis cassette; and a second inlet/outlet port of the peritoneal dialysis cassette can be fluidly connectable to a peritoneal dialysis fluid bag; the first inlet/outlet port and second inlet/outlet port fluidly connectable through a first fluid passage; wherein a first linear actuator is positioned to either occlude or allow fluid to pass through the first fluid passage.’

In any embodiment, the pneumatic system can include a pump and one or more pneumatic valves to selectively direct air to the one or more linear actuators.

In any embodiment, the pneumatic system can include one or more pressure sensors and one or more pressure controllers; the one or more pressure controllers controlling an air pressure delivered to the one or more linear actuators.

The features disclosed as being part of the second aspect can be in the second aspect, either alone or in combination, or follow any arrangement or permutation of any one or more of the described elements. Similarly, any features disclosed as being part of the second aspect can be in the first or third aspect, either alone or in combination, or follow any arrangement or permutation of any one or more of the described elements.

The third aspect relates to a method. In any embodiment, the method can use the system of the second aspect, and include the steps of: selectively directing fluid from a peritoneal dialysis fluid bag through a first inlet/outlet port of the peritoneal dialysis cassette and the one or more flow passages to a second inlet/outlet port of the peritoneal dialysis cassette and into a patient line fluidly connected to a catheter.

In any embodiment, the method can include the step of draining fluid from a patient, through the patient line, the second inlet/outlet port of the peritoneal dialysis cassette and to a drain line through a third inlet/outlet port of the peritoneal dialysis cassette.

In any embodiment, the method can include the step of selectively directing fluid from at least one peritoneal dialysis fluid source through a third inlet/outlet port of the peritoneal dialysis cassette to the first inlet/outlet port of the peritoneal dialysis cassette and to the peritoneal dialysis fluid bag prior to selectively directing fluid from the peritoneal dialysis fluid bag to the patient line.

The features disclosed as being part of the third aspect can be in the third aspect, either alone or in combination, or follow any arrangement or permutation of any one or more of the described elements. Similarly, any features disclosed as being part of the third aspect can be in the first or second aspect, either alone or in combination, or follow any arrangement or permutation of any one or more of the described elements.

### BRIEF DESCRIPTION OF THE DRAWINGS

FIGS. 1A-C illustrate a peritoneal dialysis cassette and peritoneal dialysis system.

FIGS. 2A-B illustrate a linear actuator.

FIG. 3 illustrates a pressure sensor for use with the peritoneal dialysis cassette.

FIGS. 4A-F illustrate the use of the peritoneal dialysis cassette in performing functions of the peritoneal dialysis system.

FIG. 5 is a pneumatic system operable with the linear actuators.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

An “air cushion chamber” is flexible container that expands or contracts as air is added or removed from the chamber.

The term “air pressure” refers to a force exerted by air on the walls of a container or conduit.

The term “aligned” refers to two or more components positioned with respect to each other.

A “catheter” is a flexible tube that can be inserted into a patient for adding or removing fluid.

The term “clamping” refers to a process of sealing two surfaces or components together by use of a mechanical device to squeeze the surfaces together.

The term “closed state,” when referring to a valve or linear actuator, refers to a state that blocks fluid movement through a fluid passage.

The term “comprising” includes, but is not limited to, whatever follows the word “comprising.” Use of the term indicates the listed elements are required or mandatory but that other elements are optional and may be present.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

The term “contact” refers to two or more components physically touching.

The terms “controlling” or to “control” refer to a first component causing or directing the actions of a second component.

A “control system” can be a combination of components that act together to maintain a system to a desired set of performance specifications. The control system can use processors, memory and computer components configured to interoperate to maintain the desired performance specifications. The control system can also include fluid or gas control components, and solute control components as known within the art to maintain performance specifications.

A “drain line” is a fluid line through which used or waste fluid can be disposed.

The terms “first,” “second,” and “third,” and the like, refer to separate and distinct features. For example, one or more sections can be identified as a ‘first section,” “second section,” and “third section.” Alternatively, one or more diameters can be identified as a ‘first diameter,” “second diameter,” and “third diameter.”

A “flow passage” is a conduit through which a fluid can move.

The term “fluid movement” refers to the physical movement of a fluid through a system, passage, or conduit.

The term “fluidly connectable” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

An “inlet/outlet port” is an opening or conduit through which fluids can enter or exit a component.

A “linear actuator” is a component that, when activated by pumping air into the component, extends linearly.

The term “membrane surface” refers to a flexible outer portion of a component.

The term “occlude” means to block a fluid passage, preventing fluid movement through the passage.

The term “open state,” when referring to a valve or linear actuator, refers to a state that allows fluid movement through a fluid passage.

A “patient line” refers to a fluid line fluidly connectable to a catheter for infusion of fluid into a patient or removal of fluid from a patient.

A “peritoneal dialysis cassette” is a housing containing one or more fluid passages that can be connected to fluid lines or components of a peritoneal dialysis system.

A “peritoneal dialysis cycler” or “cycler” is a component or set of components for movement of fluid into and out of the peritoneal cavity of a patient.

“Peritoneal dialysis fluid” is a dialysis solution to be used in peritoneal dialysis having specified parameters for purity and sterility. Peritoneal dialysis fluid is not the same as dialysate fluid of the type used in hemodialysis.

A “peritoneal dialysis fluid source” is a container of any type that holds components used to generate peritoneal dialysis fluid.

A “peritoneal dialysis fluid bag” is a container of any type that holds peritoneal dialysis fluid prior to infusion of the peritoneal dialysis fluid into a patient.

A “pinch bud” is an elastomeric material that contacts a membrane surface to apply force to the membrane surface.

A “pneumatic system” is a system where air is pumped through one or more fluid lines, with the air pressure used to operate one or more components.

A “pneumatic valve” is a component that either allows or blocks the movement of air through a fluid line.

A “pressure controller” is a component that can selectively limit flow of air through a conduit to maintain a specified air pressure.

The term “pressure sealing” refers to a process of sealing two surfaces or components together by applying pressure on at least one of the surfaces or components, forcing the surfaces or components together.

A “pressure sensor” is a component that can measure a force exerted by air or fluid on the walls of a container or conduit.

The term “pump” refers to any device that causes the movement of fluids or gases by applying suction or pressure.

The term “rigid surface” refers to the outer portion of a component that is substantially inflexible.

The term “sealed” refers to a fluid or airtight connection between components.

The term “selectively directing” or to “selectively direct” refer to causing fluid to move through a system in a specified pathway.

### Peritoneal Dialysis Cassette

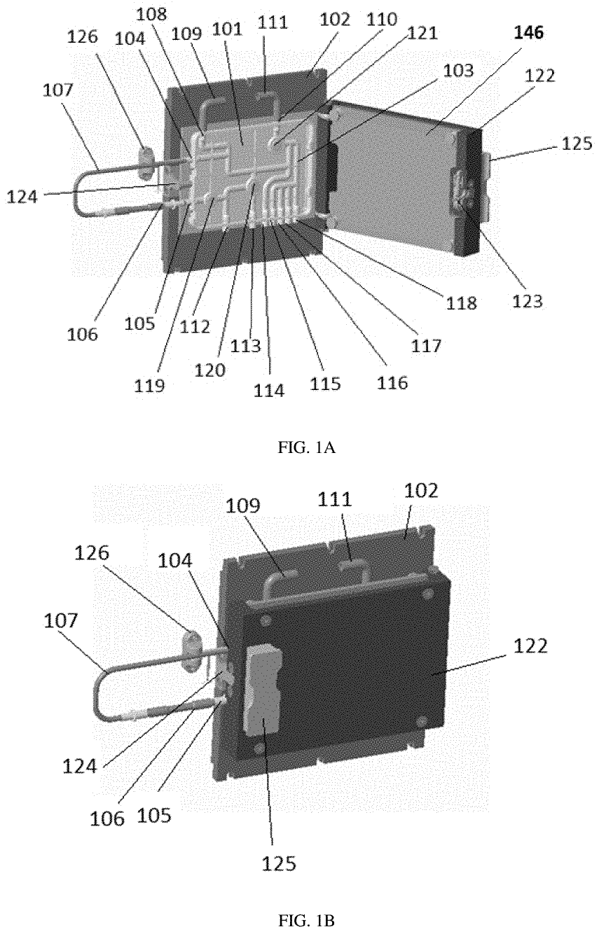

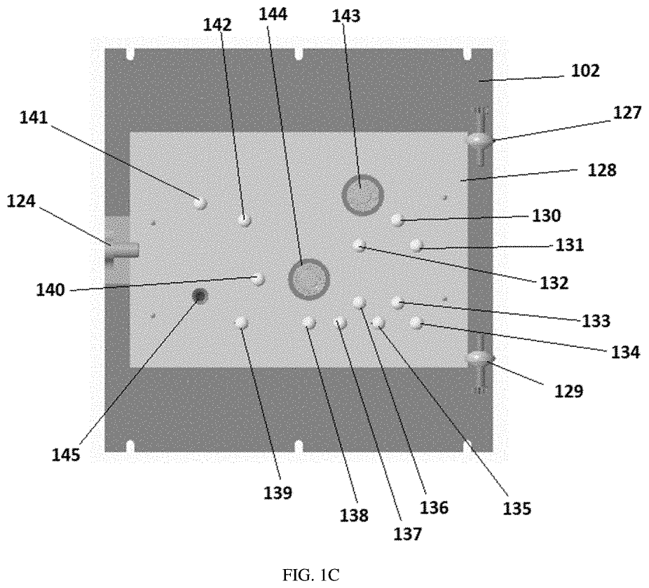

FIGS. 1A-C illustrate a peritoneal dialysis cassette 101 and a portion of a peritoneal dialysis system 102. FIG. 1A shows the peritoneal dialysis cassette 101 connected to the peritoneal dialysis system 102. FIG. 1B shows the peritoneal dialysis system 102 with a cover 122 for the peritoneal dialysis cassette 101 closed. FIG. 1C shows the portion of the peritoneal dialysis system 102 with the peritoneal dialysis cassette 101 removed.

As illustrated in FIG. 1A, the peritoneal dialysis cassette 101 can include one or more fluid passages 103 to direct fluid throughout the peritoneal dialysis system 102. One or more linear actuators (not shown in FIG. 1A) can occlude certain pathways through the fluid passages 103 to selectively direct fluid through the peritoneal dialysis cassette 101 from a first specified inlet/outlet port to a second specified inlet/outlet port. The inlet/outlet ports can each connect to fluid lines of the peritoneal dialysis system 102 to convey fluid as necessary for any function of the peritoneal dialysis system 102.

In certain embodiments, the peritoneal dialysis cassette 101 can be fluidly connected to a sensor line 107. The sensor line 107 can include one or more sensor 106 for determining one or more fluid characteristics of fluid moving through the peritoneal dialysis cassette 101. For example, sensor 106 can be a conductivity sensor for measuring the concentration of ionic solutes, a refractive index sensor for measuring the concentration of dextrose or other osmotic agents, an air bubble detector, a temperature sensor, a pressure sensor, or any other sensor. To measure the fluid characteristics, fluid can be selectively directed from inlet/outlet port 104, through sensor line 107 and sensor 106 to inlet/outlet port 105 and back into the peritoneal dialysis cassette 101. Clamp 126 or any other components can be used to fix any fluid line in place.

As illustrated in FIG. 1A, inlet/outlet port 108 can connect to fluid line 109 and inlet/outlet port 110 can connect to fluid line 111. Fluid line 109 and fluid line 111 can connect to an external pump (not shown). The pump provides the driving force for moving fluid through the peritoneal dialysis cassette 101 and the rest of the peritoneal dialysis system.

Other inlet/outlet ports can connect to different parts of the peritoneal dialysis system 102. For example, if the portion of peritoneal dialysis system 102 shown in FIG. 1A is a peritoneal dialysis cycler, inlet/outlet port 114 can connect to a peritoneal dialysis fluid bag, while fluid line 113 can connect to a patient line. Peritoneal dialysis fluid from the peritoneal dialysis fluid bag can be directed into peritoneal dialysis cassette 101 through inlet/outlet port 114, and into the patient line through inlet/outlet port 113. The linear actuators can occlude the other fluid pathways in peritoneal dialysis cassette 101, such that the peritoneal dialysis fluid is selectively directed into the patient line. Any number of inlet/outlet ports can be included in the peritoneal dialysis cassette 101 for connections between various portions of the peritoneal dialysis system 102, including a drain, water source, water purification module, peritoneal dialysis fluid generation module, peritoneal dialysis cycler, patient, pre-filled peritoneal dialysis fluid sources, and a drain or waste container. As illustrated in FIG. 1A, the peritoneal dialysis cassette 101 can include inlet/outlet port 112, inlet/outlet port 115, inlet/outlet port 116, inlet/outlet port 117, and inlet/outlet port 118. However, the peritoneal dialysis cassette 101 can include two, three, four, five, six, seven, or more inlet/outlet ports.

To install the peritoneal dialysis cassette 101, cover 122 can be opened by disengaging clasp 123 from engagement bar 124 to expose the cassette chamber. Handle 125 can be used to open or close the cover 122. Although shown as a hinged door in FIG. 1A, one of skill in the art will understand that other types of covers could be used, such as a sliding tray. The peritoneal dialysis cassette 101 can be placed into the cassette chamber and fluid lines connected to the inlet/outlet ports, as necessary. A pneumatic system (not shown) can be used to hold the peritoneal dialysis cassette 101 in place.

The peritoneal dialysis cassette 101 can include additional components, or spaces for additional components. For example, position 119 can be used for a temperature sensor. Position 120 and position 121 can be used for pressure sensors. The peritoneal dialysis cassette 101 can include a membrane surface sealed to a rigid surface, with the fluid passages 103 between the two surfaces. The membrane surface can be positioned on the side of the peritoneal dialysis cassette 101 facing the peritoneal dialysis system 102, protecting the membrane, while the rigid surface faces outwardly. The pressure sensors can also measure the fluid pressure in the fluid passages 103 through the membrane surface.

Any method can be used to seal the membrane surface to the rigid surface. In certain embodiments, the surfaces can be sealed by pressure sealing. With pressure sealing, a force is applied to one of the surfaces, forming a seal against the opposite surface sufficient to prevent leaks of fluid. Alternatively, the surfaces can be sealed by clamping. A mechanical clamp can be applied to force the surfaces together, preventing any leaks.

As described, the peritoneal dialysis cassette 101 is held in place with an air cushion. Through a pneumatic system (not shown in FIG. 1A), air is pumped into an air cushion chamber behind surface 146 of the pneumatic system. Surface 146 is pushed outward, squeezing the peritoneal dialysis cassette 101 between the peritoneal dialysis system 102 and cover 122. In certain embodiments, the membrane surface is only sealed to the rigid surface along the periphery of the peritoneal dialysis cassette 101. The air cushion also pushes the membrane surface to the rigid surface, forming the necessary seals around the fluid passages 103 in the inner portion of the peritoneal dialysis cassette 101.

FIG. 1B illustrates the peritoneal dialysis system 102 after cover 122 has been closed. The peritoneal dialysis cassette (not visible in FIG. 1B) is locked in place. As described, a pump can be fluidly connected to fluid line 109 and fluid line 111 to provide the driving force for moving fluid throughout the peritoneal dialysis system 102. Sensor line 107 is connected to inlet/outlet port 104 and inlet/outlet port 105. As described, clamp 126 can be used to hold sensor line 107 in place. Additional fluid lines can be fluidly connected to the other inlet/outlet ports (not shown in FIG. 1B) as necessary.

FIG. 1C shows the peritoneal dialysis system 102 with the peritoneal dialysis cassette removed. The cover (not shown in FIG. 1C) can connect to hinge 127 and hinge 129. The peritoneal dialysis cassette would be placed with the membrane surface facing cassette location 128. As described, linear actuators can act as valves in the peritoneal dialysis cassette. As illustrated in FIG. 1C, the system can include linear actuator 130, linear actuator 131, linear actuator 132, linear actuator 133, linear actuator 134, linear actuator 135, linear actuator 136, linear actuator 137, linear actuator 138, linear actuator 139, linear actuator 140, linear actuator 141, and linear actuator 142. FIG. 1C shows thirteen linear actuators, however, any number of linear actuators can be included depending on the needs of the system. Pressure sensor 143 and pressure sensor 144 can detect the pressure exerted on the membrane surface by fluid within the fluid passages of the peritoneal dialysis cassette. An IR probe 145 can measure the temperature of fluid within the fluid passages of the peritoneal dialysis cassette through the membrane surface.

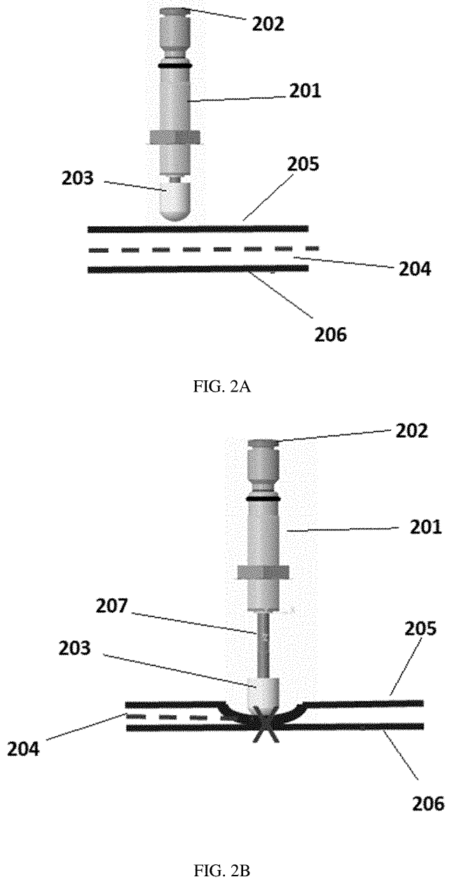

FIGS. 2A and 2B illustrate a linear actuator 201 used as a valve for the peritoneal dialysis cassette. FIG. 2A shows the linear actuator 201 in an open position, while FIG. 2B shows the linear actuator 201 in a closed position. The linear actuator 201 includes an air exhaust and entry port 202 and a pinch bud 203. As described, the peritoneal dialysis cassette includes a membrane surface 205 and a rigid surface 206 enclosing one or more fluid passages 204. The linear actuators of the system are positioned on the side of the membrane surface 205 of the peritoneal dialysis cassette. The pinch bud 203 can have a rounded or rectangular surface that does not degrade or otherwise substantially wear away membrane surface 205. The pinch bud 203 and membrane surface 205 can be made of a suitable material such as thermoplastic or hard rubber.

When the linear actuator is in an open state, as illustrated in FIG. 2A, the pinch bud 203 does not exert pressure on the membrane surface 205. The fluid passage 204 remains open, allowing fluid movement freely through the fluid passage 204. As illustrated in FIG. 2B, when air or is delivered to the linear actuator 201 through air exhaust and entry port 202, a rigid shaft 207 is extended, forcing the pinch bud 203 into the membrane surface 205 of the peritoneal dialysis cassette, placing the linear actuator 201 in a closed state. One or ordinary skill will understand that other form of linear, mechanical actuation can also be provided to linear actuator 201 to cause pinch bud 203 to contact and occlude membrane surface 205. The pinch bud 203 can push the membrane surface 205 to the rigid surface 206 occluding the fluid passage 204 and preventing fluid movement through the fluid passage 204. To return the linear actuator 201 to an open state as illustrated in FIG. 2A, the air can be exhausted through air exhaust and entry port 202 and the rigid shaft 207 retracts. A pneumatic system (not shown) in communication with a control system (not shown) can be used to control the flow of air into and out of the linear actuator 201 to selectively direct fluid through the fluid passages of the peritoneal dialysis cassette. In mechanically actuated systems, a piston or other linear force can be delivered to force the pinch bud 203 into an open and closed state in contact with membrane surface 205.

Linear actuators as illustrated in FIG. 2A-B, act with a mechanical force on the membrane surface, rather than conventional balloon activators. Using a mechanical force allows a far greater force to be applied. Further, each linear actuator can be operated by a separate pneumatic valve, making replacement of the linear actuators easier than with balloon actuators.

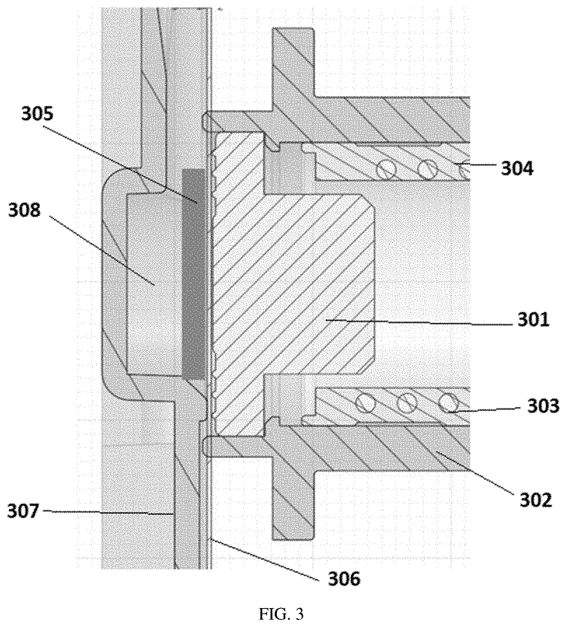

FIG. 3 is a cross-section of a non-limiting embodiment of a pressure sensor for use with the peritoneal dialysis cassette. As described, the peritoneal dialysis cassette can have one rigid surface 307 and one membrane surface 306. The two surfaces are sealed together, enclosing one or more fluid passages 308. The pressure sensor can include a sensor housing 302 containing a piezoelectric pressure sensor 301. The piezoelectric pressure sensor 301 contacts the membrane surface 306 of the peritoneal dialysis cassette at contact surface 305. The membrane surface 306 is flexible, moving outward in response to force applied by fluid moving through the fluid passage 308. The force applied by the fluid in fluid passage 306 can be detected by the electrical charge accumulated on the piezoelectric pressure sensor 301 due to the mechanical force. A control system (not shown) in communication with the pressure sensor can convert the accumulated charge to a fluid pressure. As force is applied to the pressure sensor 301 by fluid acting on the membrane surface 306, the pressure sensor can move away from the membrane surface 306 against spring 303, closing a gap 304 between the coils of the spring 303.

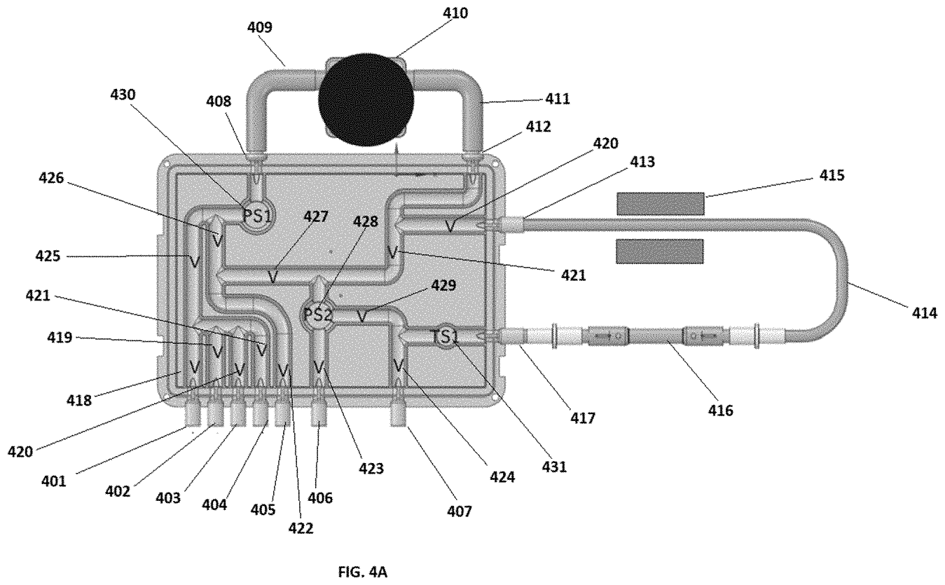

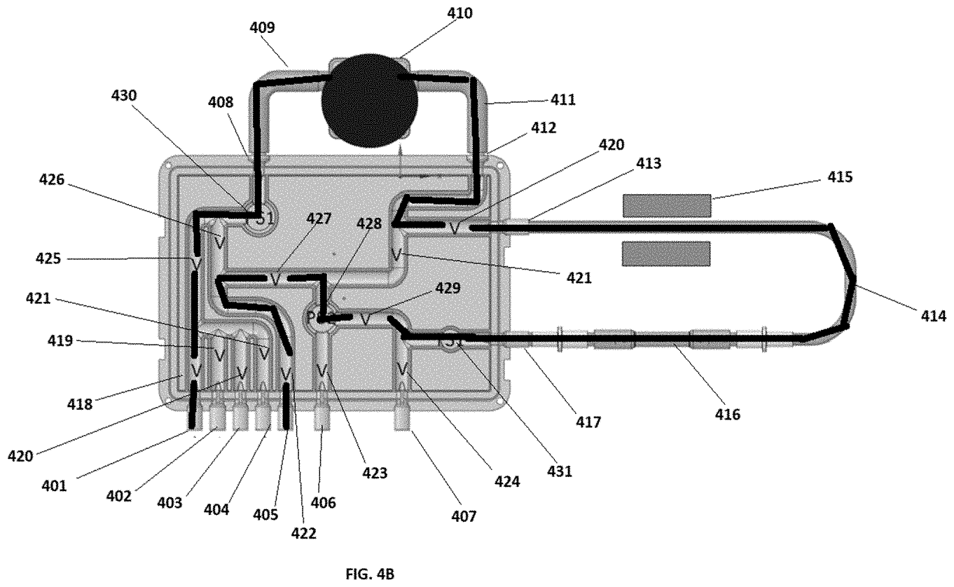

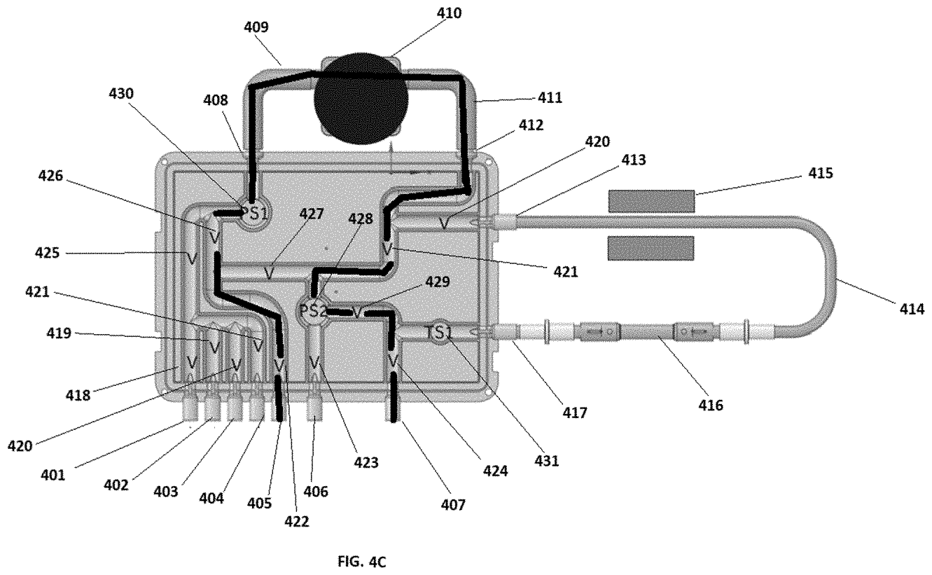

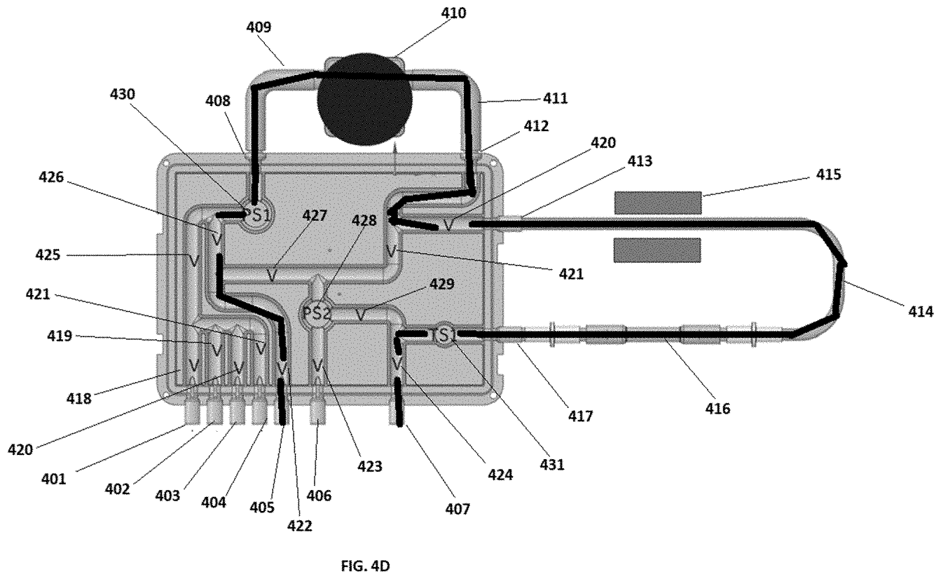

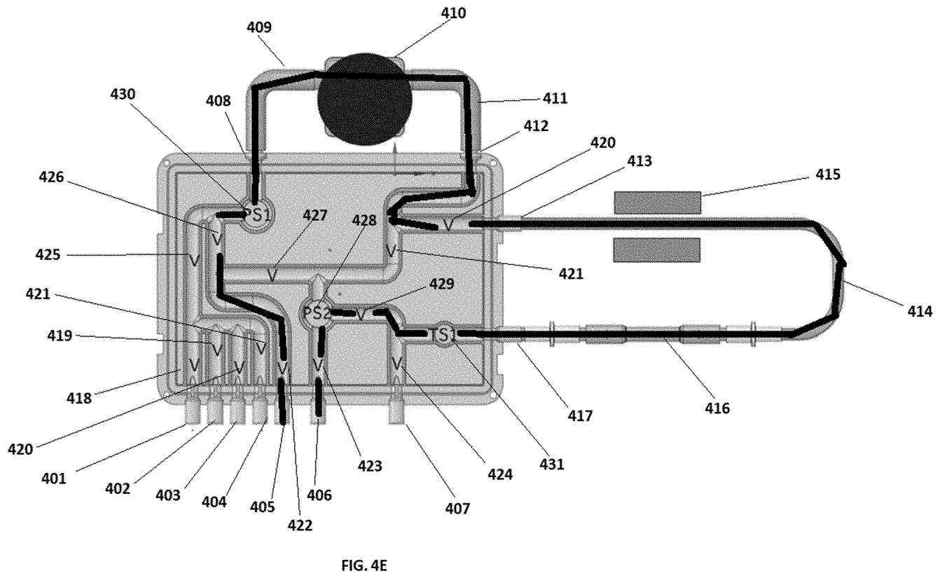

FIGS. 4A-F illustrate the use of a peritoneal dialysis cassette. FIG. 4A is the initial resting state of the peritoneal dialysis cassette, FIG. 4B shows the movement of fluid for generating peritoneal dialysis fluid from one or more constituent components, FIGS. 4C and 4D show the movement of fluid for priming the peritoneal dialysis cassette with peritoneal dialysis fluid, FIG. 4E shows the movement of fluid for filling a patient, and FIG. 4F shows the movement of fluid for draining used peritoneal dialysis fluid from the patient. In each of FIGS. 4A-F, the movement of fluid is shown in bold lines through the fluid passages of the peritoneal dialysis cassette.

As described, the peritoneal dialysis cassette can include inlet/outlet ports for connections between several components. In each of FIGS. 4A-F, inlet/outlet port 401 is fluidly connectable to the first pre-filled bag; inlet/outlet port 402 is fluidly connectable to a second pre-filled bag; inlet/outlet port 403 is fluidly connectable to a third pre-filled bag; inlet/outlet port 404 is fluidly connectable to a fourth pre-filled bag; inlet/outlet port 407 is fluidly connectable to a drain line, inlet/outlet port 405 is fluidly connectable to a peritoneal dialysis fluid bag; and inlet/outlet port 406 is fluidly connectable to a patient line. However, one of skill in the art will understand that the components each inlet/outlet port are connected to are provided for illustrative purposes only. Additional or fewer components can be connected through the peritoneal dialysis cassette and the location of each inlet/outlet port connected to each component can be changed.

In FIGS. 4A-F, the system uses four pre-filled bags as a peritoneal dialysis fluid source containing concentrated solutions of various peritoneal dialysis components. Solutions from each of the pre-filled bags are added to the peritoneal dialysis fluid bag for mixing and heating and then infused into the patient. However, the described peritoneal dialysis cassette can work with additional or fewer pre-filled bags, or can work with a single peritoneal dialysis fluid bag already having all of the components of the final peritoneal dialysis fluid.

FIG. 4A shows the peritoneal dialysis cassette in the initial state. Linear actuators are positioned on the membrane side of the peritoneal dialysis cassette to serve as valves. The peritoneal dialysis cassette illustrated in FIGS. 4A-F use 13 linear actuators serving as valve 418, valve 419, valve 420, valve 421, valve 422, valve 423, valve 424, valve 425, valve 426, valve 427, and valve 429. However, the peritoneal dialysis cassette can be arranged with more or fewer valves, depending on the needs of the system. As described, the peritoneal dialysis cassette can include position 428 and position 430 for pressure sensors and position 431 for a temperature sensor. Additional sensors can be included in sensor line 414 fluidly connected to inlet/outlet port 413 and inlet/outlet port 417, such as air bubble detector 415 and conductivity sensor 416.

FIG. 4B illustrates the use of the peritoneal dialysis cassette in filling a peritoneal dialysis fluid bag from one or more peritoneal dialysis fluid sources. Opening valve 418 and valve 425 allows a concentrate from a first concentrate source to be pumped from inlet/outlet port 401 to inlet outlet port 408 and into fluid line 409. Pump 410 provides the driving force for moving the fluid. From pump 410, the fluid is pumped through fluid line 411 and back into the peritoneal dialysis cassette through inlet/outlet port 412. Opening valve 420 while valve 421 remains closed directs the fluid through inlet/outlet port 413 and into sensor line 414. Sensor line 414 can include air bubble detector 415 and conductivity sensor 416. However, any additional or alternative sensors can be included. The fluid used in FIG. 4B is a concentrated solution of solutes used for peritoneal dialysis fluid. Conductivity sensor 416 can be used to ensure that the concentration of the solutes in the concentrated solution are within a predetermined range. From sensor line 414, the fluid can be pumped back into the peritoneal dialysis cassette through inlet/outlet port 417. Opening valve 429, valve 427, and valve 422 while keeping valve 424 closed selectively directs the fluid to inlet/outlet port 405, which can be fluidly connected to a peritoneal dialysis fluid bag (not shown). Fluid from additional concentrate sources can also be added to the peritoneal dialysis fluid bag. Closing valve 418 and opening valve 419 will allow fluid from a peritoneal dialysis fluid source connected to inlet/outlet port 402 to be added to the peritoneal dialysis fluid bag. Closing valve 419 and opening valve 420 will allow fluid from a peritoneal dialysis fluid source connected to inlet/outlet port 403 to be added to the peritoneal dialysis fluid bag. Closing valve 420 and opening valve 421 will allow fluid from a peritoneal dialysis fluid source connected to inlet/outlet port 404 to be added to the peritoneal dialysis fluid bag. Any number of peritoneal dialysis fluid sources can be added in a similar fashion.

Once the peritoneal dialysis fluid bag is filled and mixed, the peritoneal dialysis cassette can be primed with the peritoneal dialysis fluid. FIG. 4C shows the initial steps of priming the peritoneal dialysis cassette to the drain. Closing valve 418 and vale 425 from a position shown in FIG. 4B and opening valve 426 allows the pump 410 to draw fluid from the peritoneal dialysis fluid bag through inlet/outlet port 405 to inlet/outlet port 408. Closing valve 420 while opening valve 421 valve 429 allow fluid from the pump 410 to be returned through inlet/outlet port 412 to inlet/outlet port 407, which is fluidly connected to a drain line.

Once the fluid pathways that will be used for filling the patient are primed, the second priming step can be performed as illustrated in FIG. 4D. Valve 421 and valve 429 are closed while valve 420 is opened. The peritoneal dialysis fluid now travels through inlet/outlet port 413 and sensor line 414 prior to returning to the peritoneal dialysis cassette through inlet/outlet port 417. Once returned, the peritoneal dialysis fluid can again be directed to the drain through a drain line fluidly connected to inlet/outlet port 407. Selectively directing the fluid through the sensor line 414 allows the final peritoneal dialysis fluid to be checked prior to infusion into the patient. Air bubble detector 415 is used to ensure the peritoneal dialysis fluid does not contain any air bubbles. Conductivity sensor 416, as well as any other composition sensors ensure that the final peritoneal dialysis fluid is within predetermined limits for each solute. A temperature sensor at position 431 is used to ensure that the peritoneal dialysis fluid is heated to the proper temperature prior to infusion into the patient.

FIG. 4E illustrates the process of filling the patient with peritoneal dialysis fluid. Valve 424 is closed and valve 429 and valve 423 are opened. The fluid now travels from the peritoneal dialysis bag fluidly connected to inlet/outlet port 405 to a patient line fluidly connected to inlet/outlet port 406. The patient line can be connected to a catheter for infusion of the peritoneal dialysis fluid into the peritoneal cavity of the patient. While filling the patient, the fluid can still be pumped through sensor line 414 as illustrated in FIG. 4E to ensure that the composition of the peritoneal dialysis fluid remains correct and that no air bubbles are in the fluid. A temperature sensor at position 431 can be used to ensure that the peritoneal dialysis fluid remains at the proper temperature prior to infusion into the patient. A pressure sensor at position 430 and a pressure sensor at position 428 can be used to measure the fluid pressure during filling of the patient. The system can ensure that the fluid pressure is not too high during filling, and can use the fluid pressure to determine when the patient is full of peritoneal dialysis fluid.

After the peritoneal dialysis fluid has dwelled in the peritoneal cavity of the patient, the used peritoneal dialysis fluid can be drained from the patient as illustrated in FIG. 4F. Valve 422 and valve 429 are closed, while valve 427 and valve 424 are opened. Fluid can now travel from inlet/outlet port 406 fluidly connected to a patient line, through the peritoneal dialysis cassette to inlet/outlet port 407, fluidly connected to a drain line. In certain embodiments, the other valves can be controlled to selectively direct fluid from the patient through the sensor line 414, as illustrated in FIG. 4F. By directing used fluid through sensor line 414, the system can measure fluid characteristics of the used peritoneal dialysis fluid, such as the ending solute concentrations. The fluid characteristics can be used by the system or health care providers to adjust the therapy prescription as needed.

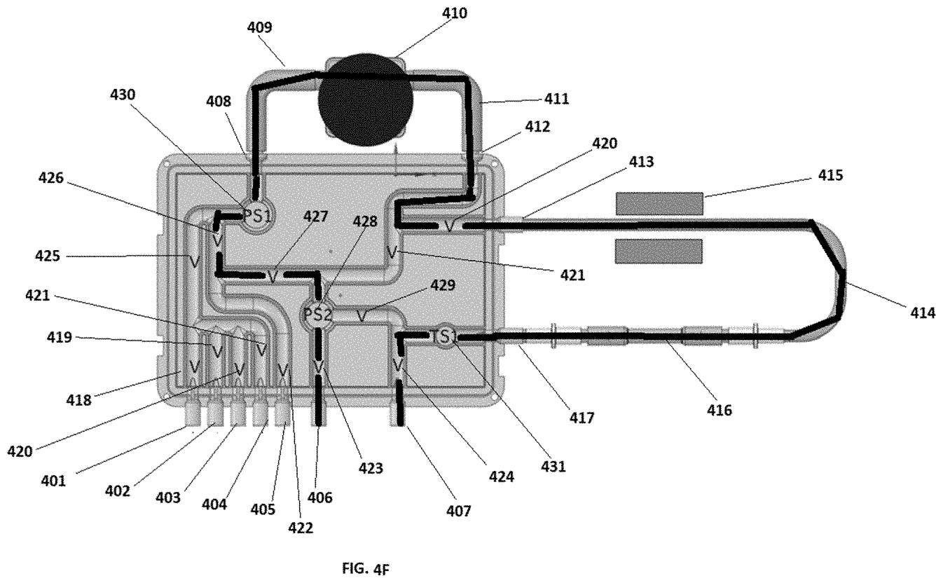

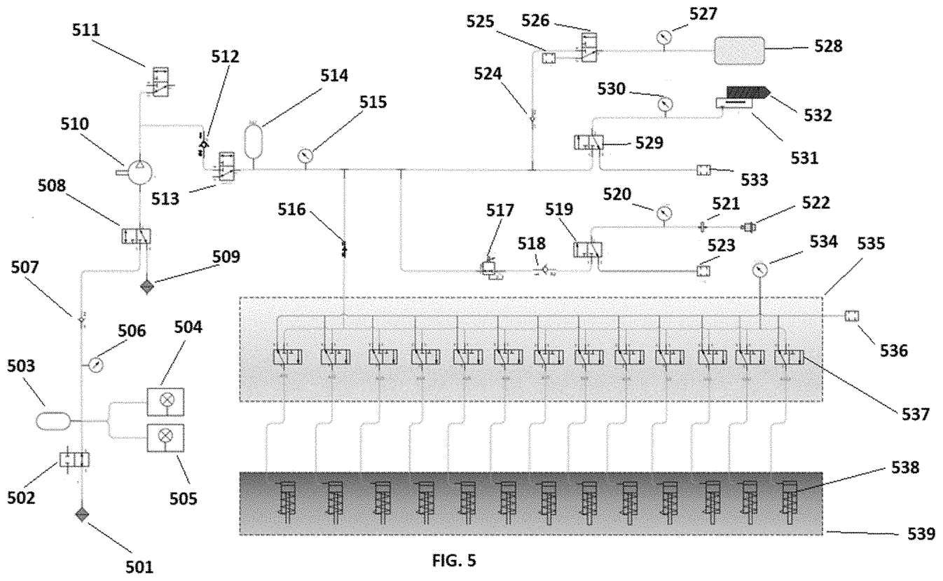

As described, the linear actuators are connected to a pneumatic system. FIG. 5 illustrates a non-limiting embodiment of a pneumatic system that can be used to operate the linear actuators, as well as other components in the peritoneal dialysis system. For clarity in FIG. 5, only a single linear actuator of the one or more linear actuators 538 of the linear actuator manifold 539 and a single pneumatic valve 537 of the pneumatic valve manifold 535 are labeled. However, as described, the system can use any number of linear actuators 538, each with an associated pneumatic valve 537.

As described, the linear actuators 538 are activated by air. Pump 510 pumps air through the system through air inlet filter 509. The air pressure can be measured by pressure sensor 506. Non-return valve 507 ensures that air cannot exit the system through air inlet filter 509. Pressure accumulator 514 accumulates pressure from the incoming air to use throughout the pneumatic system. Excess air pressure can be released through pneumatic valve 511. Pneumatic valve 508 controls the movement of air into the system from air inlet filter 509. Pump 510 pumps the air through non-return valve 512 into pressure accumulator 514 as controlled by pneumatic valve 513. Pressure from the incoming air is accumulated in pressure accumulator 514 and used to operate the linear actuators 538, as well as any other components of the peritoneal dialysis system that use air from the pneumatic system. Pressure sensor 515 measures the pressure in the lines of the pneumatic system to ensure the pressure is high enough to operate the linear actuators and other components while not exceeding the limits of the system.

To operate the pneumatic system for the linear actuators 538, air is pumped passed non-return valve 516 into pneumatic valve manifold 535. Each pneumatic valve 537 is connected to an inlet line and exhaust line. To activate a linear actuator 538, the corresponding pneumatic valve 537 is switched to allow the incoming air into the linear actuator 538 through linear actuator manifold 539. As described, when the air is pumped into the linear actuator 538, the linear actuator 538 extends, causing the pinch bud to push on the membrane surface of the peritoneal dialysis cassette, occluding a fluid passage through the cassette. When the pneumatic valve is de-activated, the linear actuator can return to an open state. Air is exhausted through the pneumatic valve manifold 535 via muffler 536 to minimize noise. Pressure sensor 534 can measure the air pressure in the pneumatic valve manifold 535 to ensure the pressure is high enough to operate the linear actuators 538 while not exceeding the limits of the system.

In certain embodiments, the pneumatic system can be used to operate additional components and functions of the peritoneal dialysis system. In FIG. 5, the pneumatic system also provides air necessary to conduct a filter integrity test. A microbial filter (not shown) can be used to ensure the sterility of peritoneal dialysis fluid. To test the integrity of the microbial filter, air is flowed into the filter and the pressure decay monitored. If the filter has a leak, the pressure in the filter will rapidly decay. To conduct the filter integrity test, pneumatic valve 519 is switched to an open state, allowing air past electronic pressure controller 517 and non-return valve 518. The electronic pressure controller 517 allows air at a specified air pressure to pass through to filter connector 522, which is connected to the microbial filter. Pressure sensor 520 measures the air pressure entering the microbial filter, and air filter 521 ensures that contaminants are not added during the test. After the microbial filter is filled with air, pneumatic valve 519 is switched to vent the air through muffler 523.

The pneumatic system can also operate a safety clamp 531. The safety clamp 531, when in a closed state, can clamp all fluid lines entering the peritoneal dialysis cassette, preventing any fluid from entering or exiting the peritoneal dialysis cassette. Pneumatic valve 529 is switched to an open state to allow air into safety clamp 531 past pressure sensor 530. When air is added to the safety clamp 531 an actuator 532 extends, clamping all fluid lines into and out of the cassette. The muffler 533 can be added in connection to pneumatic valve 529 to minimize noise.

As described, the peritoneal dialysis cassette is held in place in the dialysis system with an air cushion, which also serves to seal the membrane surface to the interior portions of the rigid surface of the cassette. Pneumatic valve 526 can be switched to an open state, allowing air past non-return valve 524 to air cushion chamber 528. Pressure sensor 527 measures the air pressure to ensure that the pressure is high enough to hold the peritoneal dialysis cassette in place and form a proper seal between the membrane surface and rigid surface. To release the air pressure and remove the peritoneal dialysis cassette, pneumatic valve 526 is switched to vent the air through muffler 525.

As described, the system can use pressure sensor 504 and pressure sensor 505 to detect the fluid pressure in the fluid channels of the peritoneal dialysis cassette. In certain embodiments, the pressure sensors can be non-contact diaphragm-based sensors, as illustrated in FIG. 3. The face of the pressure sensor is interfaced to the membrane surface of the peritoneal dialysis cassette. To ensure that the cassette membrane is mated to the pressure sensor face the system can use a vacuum, which can be generated by the pneumatic system of FIG. 5. To generate a vacuum, pneumatic valve 508 can be switched to pump air from pneumatic valve 502 rather than drawing air into the system via air inlet filter 509. Closing pneumatic valve 502 causes the pump 510 to draw air from a closed circuit, generating a vacuum between pneumatic valve 502 and pump 510. The vacuum can be applied to pressure sensor 504 and pressure sensor 505 to keep the sensors mated to the membrane surface of the peritoneal dialysis cassette. Vacuum accumulator 503 can hold the generated vacuum throughout therapy after pneumatic valve 508 is switched back to draw air into the system through air inlet filter 509. Non-return valve 507 holds the vacuum throughout the therapy process. To release the vacuum, pneumatic valve 502 can be opened to draw air in through air inlet filter 501.

Although illustrated as performing four different functions in FIG. 5, one of skill in the art will understand that the pneumatic system can be connected to any number of components that require the use of air.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A peritoneal dialysis cassette, comprising:
a rigid surface;
a membrane surface sealed to the rigid surface;
the peritoneal dialysis cassette defining one or more fluid passages in between the membrane surface and the rigid surface;
two or more inlet/outlet ports fluidly connected to the one or more fluid passages;
the one or more fluid passages aligned with one or more linear actuators; the one or more linear actuators positioned to occlude at least one of the one or more fluid passages in a closed state and to allow fluid movement through the one or more fluid passages in an open state.

2. The peritoneal dialysis cassette of claim 1, the one or more linear actuators selectively directing fluid through the one or more fluid passages from a first specified inlet/outlet port to a second specified inlet/outlet port.

3. The peritoneal dialysis cassette of claim 1, wherein the rigid surface is sealed to the membrane surface by pressure sealing or clamping.

4. The peritoneal dialysis cassette of claim 1, wherein the one or more fluid passages comprise at least a first fluid passage fluidly connecting a first inlet/outlet port to a second inlet outlet port and at least a second fluid passage fluidly connecting the first inlet/outlet port to a third inlet/outlet port.

5. The peritoneal dialysis cassette of claim 4, wherein at least one of the linear actuators is positioned to selectively direct fluid from the first inlet/outlet port to either the second inlet/outlet port or the third inlet/outlet port.

6. The peritoneal dialysis cassette of claim 4, wherein at least one of the linear actuators is positioned to selectively direct fluid from either the second inlet/outlet port or the third inlet/outlet port to the first inlet/outlet port.

7. The peritoneal dialysis cassette of claim 1, wherein each of the linear actuators comprises a pinch bud in contact with the membrane surface in the closed state.

8. A system, comprising:
the peritoneal dialysis cassette of claim 1, and
a peritoneal dialysis cycler; the peritoneal dialysis cycler comprising a pneumatic system; the pneumatic system controlling the one or more linear actuators to be in an open or a closed state.

9. The system of claim 8, further comprising a control system; the control system in communication with the pneumatic system to selectively direct fluid through the one or more fluid passages.

10. The system of claim 8, wherein the one or more linear actuators comprise at least two linear actuators.

11. The system of claim 8, wherein the peritoneal dialysis cycler comprises an air cushion chamber; the air cushion chamber in contact with the rigid surface of the peritoneal dialysis cassette.

12. The system of claim 8, wherein the peritoneal dialysis cycler comprises a patient line fluidly connectable to a catheter; the patient line fluidly connectable to a first inlet/outlet port of the peritoneal dialysis cassette; and
a second inlet/outlet port of the peritoneal dialysis cassette fluidly connectable to a peritoneal dialysis fluid bag; the first inlet/outlet port and second inlet/outlet port fluidly connectable through a first fluid passage; wherein a first linear actuator is positioned to either occlude or allow fluid to pass through the first fluid passage.

13. The system of claim 8, wherein the peritoneal dialysis cycler comprises a patient line fluidly connectable to a catheter; the patient line fluidly connectable to a first inlet/outlet port of the peritoneal dialysis cassette; and
a second inlet/outlet port of the peritoneal dialysis cassette fluidly connectable to a drain line; the first inlet/outlet port and second inlet/outlet port fluidly connectable through a first fluid passage; wherein a first linear actuator is positioned to either occlude or allow fluid to pass through the first fluid passage.

14. The system of claim 8, wherein the peritoneal dialysis cycler comprises at least one peritoneal dialysis fluid source fluidly connected to a first inlet/outlet port of the peritoneal dialysis cassette; and
a second inlet/outlet port of the peritoneal dialysis cassette fluidly connectable to a peritoneal dialysis fluid bag; the first inlet/outlet port and second inlet/outlet port fluidly connectable through a first fluid passage; wherein a first linear actuator is positioned to either occlude or allow fluid to pass through the first fluid passage.

15. The system of claim 8, wherein the pneumatic system comprises a pump and one or more pneumatic valves to selectively direct air to the one or more linear actuators.

16. The system of claim 15, wherein the pneumatic system further comprises one or more pressure sensors and one or more pressure controllers; the one or more pressure controllers controlling an air pressure delivered to the one or more linear actuators.

17. A method using the system of claim 8, comprising the steps of:
selectively directing fluid from a peritoneal dialysis fluid bag through a first inlet/outlet port of the peritoneal dialysis cassette and the one or more flow passages to a second inlet/outlet port of the peritoneal dialysis cassette and into a patient line fluidly connected to a catheter.

18. The method of claim 17, further comprising the step of draining fluid from a patient, through the patient line, the second inlet/outlet port of the peritoneal dialysis cassette and to a drain line through a third inlet/outlet port of the peritoneal dialysis cassette.

19. The method of claim 17, further comprising the step of selectively directing fluid from at least one peritoneal dialysis fluid source through a third inlet/outlet port of the peritoneal dialysis cassette to the first inlet/outlet port of the peritoneal dialysis cassette and to the peritoneal dialysis fluid bag prior to selectively directing fluid from the peritoneal dialysis fluid bag to the patient line.
